(19) United States
(12) Patent Application Publication
(10) Pub. No.: US 2017/0014730 A1
(43) Pub. Date: Jan. 19, 2017

(54) COLUMN FOR THERMAL TREATMENT OF FLUID MIXTURES, ESPECIALLY THOSE COMPRISING (METH)ACRYLIC MONOMERS

(71) Applicant: BASF SE, Ludwigshafen (DE)

(72) Inventors: Ulrich HAMMON, Mannheim (DE); Thomas Walter, Hassloch (DE)

(73) Assignee: BASF SE, Ludwigshafen (DE)

(21) Appl. No.: 15/210,220

(22) Filed: Jul. 14, 2016

Related U.S. Application Data
(60) Provisional application No. 62/193,611, filed on Jul. 17, 2015.

(30) Foreign Application Priority Data
Jul. 17, 2015 (DE) 10 2015 213 493.8

(51) Int. Cl. B01D 3/32; C07C 51/44; B01D 3/26

(52) U.S. Cl. CPC B01D 3/32; B01D 3/324; B01D 3/26; C07C 51/44

(57) ABSTRACT

The present invention relates to a column (1) for thermal treatment of fluid mixtures, having a cylindrical, vertical column body (2) which forms a column cavity (3), a plurality of trays (8) mounted in the column cavity (3) and spaced apart vertically from one another, at least one stub (11) disposed within the column body (2) and extending away from the column body (2), and a closable inspection orifice (9) formed in the stub (11). The characteristic feature of the column of the invention is that a spray device (20) disposed in the column body (2) can spray liquid (22) at least against the surface (15) of the stub (11) directed into the column cavity (3).

The present invention relates to a column of thermal treatment of fluid mixtures. It has a cylindrical, vertical column body which forms a column cavity. The common further comprises a plurality of trays mounted in the column cavity and spaced apart vertically from one another. In addition, the column comprises at least one stub disposed within the column body and extending from the column body, and a closable inspection orifice formed in the stub. The column is especially a separating column. The invention further relates to a thermal separation process between at least one gas ascending within a column and at least one liquid descending within the column.

In separating columns, gaseous (ascending) and liquid (descending) streams are in many cases conducted in countercurrent, at least one of the streams especially comprising a (meth)acrylic monomer. As a result of the inequilibria that exist between the streams, heat and mass transfer takes place, which ultimately causes the removal (or separation) desired in the separating column. In this document, such separating processes shall be referred to as thermal separating processes.

Examples of, and hence elements of, the expression “thermal separating processes” used in this document are fractional condensation (cf., for example, DE 19924532 A1, DE 10243625 A1 and WO 2008/090190 A1) and rectification (in both cases, ascending vapor phase is conducted in countercurrent to descending liquid phase; the separating action is based on the vapor composition at equilibrium being different from the liquid composition), absorption (at least one ascending gas is conducted in countercurrent to at least one descending liquid; the separating action is based on the different solubility of the gas constituents in the liquid) and desorption (the reverse process of absorption; the gas dissolved in the liquid phase is removed by lowering the partial pressure; if the partial pressure of the material dissolved in the liquid phase is lowered at least partly by passing a carrier gas through the liquid phase, this thermal separating process is also referred to as stripping; alternatively or additionally (simultaneously as a combination), the lowering of the partial pressure can also be brought about by lowering the working pressure).

For example, the removal of (meth)acrylic acid and/or (meth)acrolein from the product gas mixture of the catalytic gas phase oxidation can be conducted in such a way that the (meth)acrylic acid and/or the (meth)acrolein is first subjected to basic removal by absorption into a solvent (e.g. water or an organic solvent) or by fractional condensation of the product gas mixture, and the absorbate or condensate obtained is subsequently separated further to obtain (meth)acrylic acid and/or (meth)acrolein of greater or lesser purity (cf., for example, DE-10332758 A1, DE 10243625 A1, WO 2008/090190 A1, DE 10336386 A1, DE 19924532 A1, DE 19924533 A1, DE 102010001228 A1, WO 2004/035514 A1, EP 1125912 A2, EP 982289 A2, EP 982287 A1 and DE 10218419 A1).

The notation “(meth)acrylic monomers” in this document is an abbreviated form of “acrylic monomers and/or methacrylic monomers”.

The term “acrylic monomers” in this document is an abbreviated form of “acrolein, acrylic acid and/or esters of acrylic acid”.

The term “methacrylic monomers” in this document is an abbreviated form of “methacrolein, methacrylic acid and/or esters of methacrylic acid”.

In particular, the (meth)acrylic monomers addressed in this document shall comprise the following (meth)acrylic esters: hydroxyethyl acrylate, hydroxyethyl methacrylate, hydroxypropyl acrylate, hydroxypropyl methacrylate, glycidyl acrylate, glycidyl methacrylate, methyl acrylate, methyl methacrylate, n-butyl acrylate, isobutyl acrylate, isobutyl methacrylate, n-butyl methacrylate, tert-butyl acrylate, tert-butyl methacrylate, ethyl acrylate, ethyl methacrylate, 2-ethylhexyl acrylate, 2-ethylhexyl methacrylate, N,N-dimethylaminoethyl acrylate and N,N-dimethylaminoethyl methacrylate.

(Meth)acrylic monomers are important starting compounds for preparation of polymers which find use, for example, as adhesives or as water-superabsorbing materials in hygiene articles.

On the industrial scale, (meth)acrolein and (meth)acrylic acid are prepared predominantly by catalytic gas phase oxidation of suitable C₃/C₄ precursor compounds (or of precursor compounds thereof). In the case of acrolein and acrylic acid, such precursor compounds used are preferably propene and propane. In the case of methacrylic acid and of methacrolein, isobutene and isobutane are preferred precursor compounds.

As well as propene, propane, isobutene and isobutane, however, suitable starting materials are also other compounds comprising 3 or 4 carbon atoms, for example isobutanol, n-propanol or precursor compounds thereof, for example the methyl ether of isobutanol. Acrylic acid can also be obtained by oxidation of acrolein under gas phase catalysis. Methacrylic acid can also be obtained by oxidation of methacrolein under gas phase catalysis.

In the context of such preparation processes, it is normal to obtain product mixtures from which the (meth)acrylic acid and/or the (meth)acrolein have to be removed.

Esters of (meth)acrylic acid are obtainable, for example, by direct reaction of (meth)acrylic acid and/or (meth)acrolein with the appropriate alcohols. However, in this case too, product mixtures are at first obtained, from which the (meth)acrylic esters have to be removed.

The separating columns in which these separating processes are conducted comprise separating internals. In the thermal separating processes, these have the purpose of increasing the surface area for the heat and mass transfer which brings about the separation in the separating column (“the transfer area”).

Useful internals of this kind include, for example, structured packings, random packings and/or trays, which are also referred to as mass transfer trays. Frequently, separating columns used are those which comprise at least one sequence of mass transfer trays as a portion of the separating internals.

The purpose of mass transfer trays is to provide areas having essentially continuous liquid phases in the separating column in the form of liquid layers that form thereon.

The surface of the vapor and/or gas stream which ascends within the liquid layer and is distributed in the liquid phase is then the crucial transfer area.

A sequence of mass transfer trays is understood to mean a sequence (a succession) of at least two mass transfer trays generally of the same design (i.e. identical), arranged one above another in the separating column. Advantageously for application purposes, the clear distance between two immediately successive mass transfer trays in such a series (sequence) of mass transfer trays is uniform (meaning that the mass transfer trays are arranged equidistantly one above another in the separating column).

The simplest embodiment of a mass transfer tray is called a trickle sieve tray. This comprises a plate, or plate segments joined to form a plate, having essentially planar passage orifices, for example round holes and/or slots, for the ascending gas or vapor phase (the terms “gaseous” and “vaporous” are used synonymously in this document) distributed over the plate (cf., for example, DE 10230219 A1, EP 1279429 A1,U.S. Pat. No. 3,988,213 and EP 1029573 A1). Any orifices beyond these (for example at least one downcomer (at least one drain segment)) are generally not present in trickle sieve trays. As a result of this absence of downcomers, both the gas ascending within the separating column (the vapor ascending within the separating column) and the liquid descending within the separating column have to move, flowing in opposite directions, alternating in time, through the (same) passage orifices (through the open cross sections of the passages). Reference is also made to the “dual flow” of ascending gas and descending liquid through the passage orifices, which is the reason why the literature frequently also uses the term “dual-flow trays” for mass transfer trays of this type.

The cross section of the passage orifices of a dual-flow tray is matched in a manner known per se to the load thereon. If the cross section is too small, the ascending gas passes through the passage orifices at such a high velocity that the liquid descending within the separating column is entrained essentially without separating action. If the cross section of the passage orifices is too great, ascending gas and descending liquid move past one another essentially without exchange, and the mass transfer tray is at risk of running dry.

In other words, the separation-active working range of a trickle sieve tray (dual-flow tray) has two limits. There has to be a minimum limiting velocity of the ascending gas, in order that a certain liquid layer is held on the trickle sieve tray, in order to enable separation-active working of the trickle sieve tray. The upper limit in the velocity of the ascending gas is fixed by the flood point, when the gas velocity leads to backup of the liquid on the trickle sieve tray and prevents it from trickling through.

The longest dimension of the passage orifices of an industrial dual-flow tray (=longest direct line connecting two points on the outline of the passage orifice cross section) is typically 10 to 80 mm (cf., for example, DE 10156988 A1). Normally, the passage orifices are identical within a trickle sieve tray (in other words, they all have the same geometric shape and the same cross section (the same cross-sectional area)). Appropriately in application terms, the cross-sectional areas are circles. In other words, preferred passage orifices of trickle sieve trays are circular holes. The relative arrangement of the passage orifices of a trickle sieve tray advantageously follows a strict triangular pitch (cf., for example, DE 10230219 A1). It is of course also possible for the passage orifices to be configured differently within one and the same trickle sieve tray (to vary over the trickle sieve tray).

Advantageously in application terms, a sequence of trickle sieve trays comprises trickle sieve trays of the same design (identical trickle sieve trays) in a separating column, preferably arranged equidistantly one above another.

According to DE 10156988 A1, it is also possible to employ sequences of trickle sieve trays in separating columns having a uniform (preferably circular) cross section within a dual-flow tray, but varying within the sequence (for example decreasing from the bottom upward).

In general, each dual-flow tray in a corresponding tray sequence concludes flush with the wall of the separating column. However, there are also embodiments in which an intermediate space interrupted only partly by bridges exists between the column wall and tray. Aside from the actual passage orifices, a trickle sieve tray typically has, at most, orifices which serve to secure the tray on support rings or the like (cf., for example, DE 10159823 A1).

Within the normal working range of a sequence of trickle sieve trays, the liquid descending within the separating column trickles downward in droplets from dual-flow tray to dual-flow tray, meaning that the gas phase ascending between the dual-flow trays is permeated by a divided liquid phase. Some of the droplets that hit the lower trickle sieve tray in each case are atomized. The gas stream flowing through the passage orifices bubbles through the liquid layer formed on the surface of the tray, with intense heat and mass transfer between the liquid and the gas.

According to the gas and liquid load, there is a tendency in trickle sieve trays, in the case of column diameters of >2 m, for slightly unequal distributions of liquids to build up, and thus for the liquid hold-up of a tray to vary over a large area or for a circulating wave to form, which can firstly adversely affect the mechanical stability of the column body and secondly reduces the separating action, since the liquid distribution under these conditions is then time-dependent and highly location-dependent. To avoid such non-steady states, it has therefore been found to be advantageous to distribute baffles in the form of vertical metal sheets over the tray cross section, which prevent or at least greatly reduce buildup of liquid within the column body. The height of the metal sheets should correspond approximately to the height of the liquid froth layer that forms. This is typically about 20 cm at customary loads.

The cross section of a separating column is generally circular. This applies correspondingly to the accompanying mass transfer trays.

Dual-flow trays usable for the purposes of this document are described, for example, in Technische Fortschrittsberichte [Technical Progress Reports], vol. 61, Grundlagen der Dimensionierung von Kolonnenboden [Fundamentals of the Dimensioning of Column Trays], pages 198 to 211, Verlag Theodor Steinkopf, Dresden (1967) and in DE 10230219 A1.

The above-described sequence of trickle sieve trays which comprises mass transfer trays without forced flow of the liquid descending onto the tray on the tray is distinguished from sequences of mass transfer trays with such forced liquid flow.

It is a characteristic feature of these mass transfer trays that they additionally have, as well as the passage orifices already described, at least one downcomer. This is at least one downflow orifice present in the mass transfer tray, toward which the liquid which has descended onto the mass transfer tray (for example over an outlet weir (in the simplest embodiment, this may be an upward extension of the downflow orifice with a neck (a chimney; in the case of a circular downflow orifice, a tube))) flows, and which runs into a shaft which feeds the mass transfer tray below in the sequence and which is generally configured with central symmetry with respect to an axis pointing in the longitudinal direction of the column. The cross section of the shaft may vary (for example narrow) along this axis or else be constant.

By virtue of the at least one downcomer of the mass transfer tray, within a sequence of such mass transfer trays, the liquid descending from a higher mass transfer tray can descend independently of the gas or vapor which continues to rise through the passage orifices of this mass transfer tray as at least one feed of liquid to the next lowest mass transfer tray of the sequence.

The essential basis for this separation of the flow paths of descending liquid and ascending gas is the hydraulic seal (the liquid seal or else shaft seal) of the respective downcomer for the ascending gas (a downcomer must not form a bypass past the passage orifices for the ascending gas; the gas stream (the vapor stream) must not ascend past the passage orifices through a downcomer).

Such a hydraulic seal can be achieved, for example, by drawing the downcomer downward (allowing it to run downward) to such an extent that it is immersed deeply enough into the liquid layer on the next lowest mass transfer tray of the sequence (such a seal is also referred to in this document as “static seal”). The liquid level needed for this purpose can be achieved on the lower mass transfer tray, for example, through the height of appropriate outlet weirs.

However, such a design has the disadvantage that the area of the lower mass transfer tray directly below the outflow cross section of a downcomer of the mass transfer tray above (called the feed area) cannot have any passage orifices for the ascending gas and so is not available for heat and mass transfer between the liquid layer formed on the lower mass transfer tray and the ascending gas.

In an alternative embodiment, the lower outflow end of the downcomer is truncated to such an extent that it is no longer immersed into the liquid layer present on the mass transfer tray below. In this case, between the lower end of the at least one downcomer and the mass transfer tray onto which the downcomer runs, a sufficiently large intermediate space remains, in which a froth layer forms and heat and mass transfer can take place between a liquid layer which accumulates (on the lower mass transfer tray) and a gas ascending (through this tray). In other words, in this case, the “feed area” of the at least one downcomer on the mass transfer tray below may also have passage orifices and can thus increase the available exchange area of the mass transfer tray, and hence the separating action thereof.

A static liquid seal of the downcomer can be brought about in this case, for example, with the aid of a collecting cup mounted below the outflow end of the downcomer. Appropriately in application terms, in this case, the outer wall of the collecting cup is truncated to such an extent that the outflow end of the downcomer is immersed into the collecting cup (it is also possible to allow the lower edge of the downcomer to end at the upper edge of the collecting cup). In the course of operation of the column, the liquid flowing downward through the downcomer collects in the collecting cup until it flows over the upper edge of the outer wall of the collecting cup. The lower edge of the downcomer is immersed into the liquid present in the collecting cup, and the collecting cup forms a siphon-like liquid seal of the downcomer.

Alternatively, a truncated downcomer can also be sealed dynamically. For this purpose, the downcomer can be sealed, for example, at the lower end thereof with a tray provided with exit orifices of such dimensions that the liquid is backed up in the downcomer and prevents the penetration of gas (cf., for example, EP 0882481 A1 and DE 10257915 A1). The shaft seal is established in this case dynamically through the pressure drop which arises at the exit orifices. In other words, in the case of static sealing, the downcomer is sealed by virtue of the outflow end thereof being immersed into backed-up liquid, and, in the case of dynamic sealing, construction features at the outflow end of the downcomer have the effect that the exiting liquid suffers a pressure drop which brings about backup of the liquid descending in the downcomer, which causes the seal. In the simplest case, such a pressure drop can be caused by virtue of a small cross section of the exit orifice of the downcomer being selected compared to the mean cross section of the shaft.

For separation-active operation of a sequence of such mass transfer trays, the design of the at least one downcomer is relevant. Firstly, the cross section of the at least one downcomer selected must be sufficiently large (in general, the corresponding cross-sectional area is greater than the cross-sectional area of a passage orifice), in order that the liquid, even at maximum loading of the separating column, can still descend reliably through the at least one downcomer therewith, and does not back up on the tray above. On the other hand, it has to be ensured that, even in the case of minimal liquid loading, the hydraulic seal of the at least one downcomer still exists.

At a low gas loading, there is likewise the risk of liquid trickling through the passage orifices. In addition, the liquid has to be able to back up in a downcomer to such an extent that the weight of the backed-up liquid column is sufficient to convey the liquid into the gas space below the mass transfer tray to which the downcomer is connected. This backup height determines the required minimum length of the downcomer and thus partly determines the tray separation required in a sequence of corresponding mass transfer trays. A significant partial determining factor for the above backup height (backup length) is the pressure drop ΔP of a mass transfer tray. This pressure drop is suffered by the ascending gas as it flows through the passage orifices, and the “hydrostatic” head of the froth layer on the mass transfer tray. It is responsible for the fact that the pressure in the gas phase of a sequence of such mass transfer trays increases from the top downward. For the “hydrostatic” pressure hₚ of the liquid backed up in the downcomer of a mass transfer tray, it is therefore necessary for at least the condition hₚ>ΔP of the mass transfer tray to be met. These connections are also known to the person skilled in the art, for example, from EP 1704906 A1, as is the possibility of ensuring that, with an inflow weir on the lower mass transfer tray, in the case of static sealing of the downcomer of the upper mass transfer tray in the liquid layer on the lower mass transfer tray, the shaft seal still exists even in the case of low loading with descending liquid. However, the use of an inflow weir increases the backup height required in the downcomer to force the liquid backed up therein onto the lower mass transfer tray. Overall, the element of the downcomer enables a broadening of the separation-active working range compared to the trickle sieve tray. A favorable outflow velocity of the liquid backed up in the downcomer from the downcomer in the process according to the invention is, for example, 1.2 m/s.

In addition, it enables forced circulation of the liquid descending onto a mass transfer tray on this tray.

If, for example, only half of a (preferably circular) mass transfer tray has at least one downcomer (which means that all downflow orifices are present with their full extent within the corresponding circle segment), and, in a sequence of at least two identical mass transfer trays of this kind, the mass transfer trays in a separating column are arranged one on top of another such that two mass transfer trays in the separating column, one of which follows the other in the downward direction, are each mounted offset (turned) by 180° relative to one another about the longitudinal axis of the column, such that the downcomers thereof are on opposite sides (in opposite halves) of the separating column, the liquid which descends from an upper mass transfer tray through the at least one downcomer thereof to the mass transfer tray mounted below must necessarily (i.e. of necessity) flow on this lower mass transfer tray, viewed over the lower mass transfer tray, from the at least one feed area of the at least one downcomer of the upper mass transfer tray (that mounted above) (from the at least one feed through the at least one downcomer of the upper mass transfer tray) to the at least one downcomer of this lower mass transfer tray. In other words, the liquid descending from the upper to the lower tray is inevitably conducted across the tray from the at least one feed to the at least one outlet.

Such a liquid flow on a mass transfer tray within a sequence of identical mass transfer trays shall be referred to in this document as a crossflow, the sequence of such identical mass transfer trays as a sequence of identical crossflow mass transfer trays, and the individual mass transfer trays within the sequence as crossflow mass transfer trays.

In the simplest case, the crossflow mass transfer tray is a crossflow sieve tray. Apart from the at least one downcomer, it has passage orifices for the gas ascending in a separating column, and useful embodiments for the configuration thereof are in principle all of those addressed for the trickle sieve tray. A crossflow sieve tray preferably likewise has circular holes as passage orifices, and these likewise, advantageously for application purposes, have a uniform radius. As already mentioned, the at least one downcomer enables the liquid descending in a separating column, in a sequence of crossflow sieve trays, irrespective of the flow path of the vapor ascending in the sequence, to descend (through the passage orifices) from a higher crossflow sieve tray to the next lowest crossflow sieve tray. On the lower tray, the liquid flows in crosscurrent from the at least one feed of the lower tray, which is formed by the at least one outlet of the higher crossflow sieve tray, to the at least one downcomer (to the at least one outlet) of the lower tray, the desired liquid height on the lower crossflow sieve tray being partly ensured, for example, by the height of at least one outlet weir over which the liquid can flow to the at least one downcomer. In addition, the liquid is retained on the crossflow sieve tray by the backup pressure of the vapor ascending in the separating column. If the vapor loading of a crossflow sieve tray, however, falls below a minimum value, the result may be trickling of the liquid through the passage orifices, which reduces the separating action of the crossflow sieve tray and/or leads to the crossflow sieve tray running dry.

This risk of running dry can be counteracted by providing the downflow orifice of the at least one downcomer with an outlet weir and extending the respective passage orifice in the upward direction with a neck (a chimney; in the case of a circular passage orifice, a tube).

Normally mounted over the end of the neck are vapor-deflecting hoods (bubble caps, inverted cups) (these may in the simplest case be placed on with screw connections to the neck (for example at the front and back) and are effectively pulled over the neck), which are immersed into the liquid backed up on the tray. The vapor ascending through the respective passage orifice at first flows through the neck thereof into the accompanying hood, in which it is deflected, in order then, in contrast to the crossflow sieve tray, to flow in parallel to the tray surface from the hood into the liquid backed up thereon (such a “parallel outflow” is generally favorable in processes according to the invention in that it is able to “blow away” undesirably formed polymer particles and thus to bring about a self-cleaning effect). The gas streams (vapor streams) exiting from adjacent hoods, preferably distributed equidistantly over the trays, agitate the liquid backed up on the tray and form a froth layer therein, in which the heat and mass transfer takes place. Such crossflow mass transfer trays are also referred to as crossflow bubble-cap trays or crossflow hood trays. Since they have backed-up liquid even in the case of low loading with ascending gas (vapor) and thus are at no risk of running dry, they are also referred to as hydraulically sealed crossflow trays. Compared to crossflow sieve trays, they typically require higher capital costs and cause higher pressure drops of the gas ascending through them. The passage orifice of these trays designed (configured) as described is also referred to as bubble-cap passage orifice or hood passage orifice, in contrast to the simple sieve passage orifice of a sieve tray.

The most important component of the crossflow bubble-cap tray is the bubble cap (cf., for example, DE 10243625 A1 and Chemie-Ing.-Techn. Volume 45, 1973/No. 9+10, p. 617 to 620). According to the configuration and arrangement of the bubble caps (vapor deflecting hoods, hoods), crossflow bubble-cap trays are divided, for example, into crossflow round bubble-cap trays (the cross sections of passage orifice, chimney (neck) and bubble cap (vapor deflecting hood) are round (for example the cylinder bubble-cap tray or the flat bubble-cap tray), tunnel crossflow trays (the cross sections of passage orifice, chimney and bubble cap (hood) are rectangular; the passages with their bubble caps are arranged one after another within rows arranged alongside one another, with the longer rectangular edge aligned parallel to the crossflow direction of the liquid) and crossflow Thormann® trays (the cross sections of passage orifice, chimney and bubble cap (hood) are rectangular; the passages with their bubble caps are arranged one after another within rows arranged alongside one another, with the longer rectangular edge aligned at right angles to the crossflow direction of the liquid). Crossflow Thormann trays are described, for example, in DE 19924532 A1 and in DE 10243625 A1, and the prior art acknowledged in these two documents.

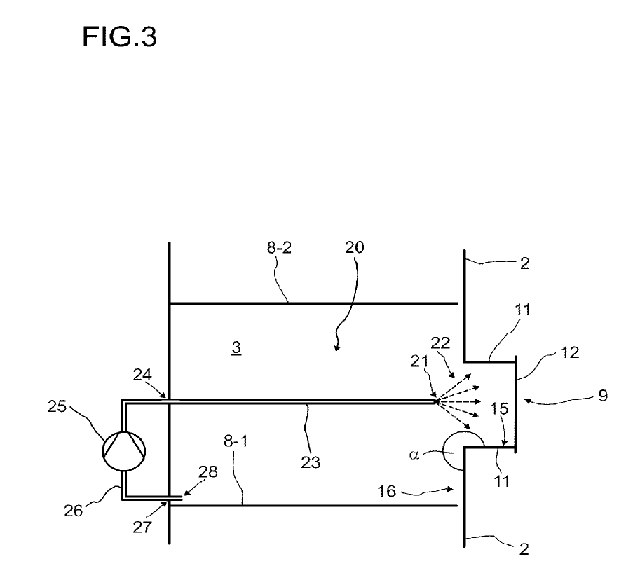

The bubble-cap edge in crossflow bubble-cap trays may have very different forms (cf. DE 10243625 A1 and Chemie-Ing. Techn. Volume 45, 1973/No. 9+10, p. 617 to 620). FIG. 3 from Chemie-Ing. Techn. Volume 45, 1973/No. 9+10, p. 618 shows some examples of the serrated and slotted edge. The serrations and slots are typically shaped such that the vapor emerging from the bubble cap into the liquid backed up on the mass transfer tray dissolves very easily into a large number of bubbles or vapor jets. The above FIG. 3 and various figures in DE 10243625 A1 also show illustrative embodiments of bubble-cap edges having a sawtooth-like structure, the teeth of which are additionally equipped with guide fins (guide surfaces) (“slots bent open”). The guide fins are intended to impose a tangential exit direction on the gas stream (vapor stream) exiting from the sawtooth-like slots bent open (direct the gas exit into the liquid in an oblique direction), as a result of which the surrounding liquid receives a directed movement pulse which, in cooperation with the arrangement of the bubble caps (vapor deflecting hoods), can lead to a directed liquid flow on the crossflow bubble-cap tray, which is superimposed on the crossflow which is established, viewed over the mass transfer tray (frequently, such slots bent open are also referred to as forcing slots). For example, in a sequence of crossflow Thormann trays, the liquid on a lower crossflow Thormann tray does not flow directly across the tray, but rather, in the manner described above, is driven in a meandering manner from the at least one feed to the at least one outlet. The space between two hoods of a crossflow Thormann tray arranged one after the other in crossflow direction forms a channel in each case, in which the liquid flows. The detailed configuration of a crossflow Thormann tray is additionally normally in such a manner that the liquid flows in countercurrent in two channels which are successive in each case in crossflow direction (cf., for example, FIG. 3 of DE 10243625 A1). The meandering crossflow which results in this manner prolongs the flow path of the liquid from the at least one feed to the at least one outlet, which promotes the separating action of a crossflow Thormann tray.

As already stated, in a crossflow bubble-cap tray, the gas emerging from the bubble cap, in contrast to the crossflow sieve tray, is introduced parallel to the tray surface into the liquid backed up on the crossflow bubble cap tray. Frictional and buoyancy forces ensure that, with increasing distance of the emerging gas stream from the bubble-cap edge, more and more substreams thereof are deflected in a direction at right angles to the crossflow bubble-cap tray and ultimately escape from the liquid layer. With increasing gas loading of a bubble cap, the velocity of the gas stream emerging from it grows, which increases the distance from the edge of the bubble cap (“the effective range of the bubble cap”) up to which the above-described deflection occurs.

This dependence of the effective range of a rigid bubble cap on the gas loading can be counteracted by configuring (designing) the passage orifice of a crossflow mass transfer tray as a valve (as a valve passage orifice). The resulting crossflow mass transfer trays are referred to as crossflow valve trays (cf., for example, DD 279822 A1, DD 216633 A1 and DE 102010001228 A1).

The term “crossflow valve trays” in this document thus covers crossflow mass transfer trays which have passage orifices (tray holes) with limited-stroke plate, ballast or lifting valves (floating flaps) which adjust the size of the vapor passage orifice to the respective column loading.

In a simple configuration, the passage orifices of the tray are covered for the aforementioned purpose with covers or plates (disks) movable in the upward direction.

In the course of passage of the ascending gas, the lids (plates, disks) are raised by the gas stream in a corresponding guide structure (guide cage) additionally mounted over the respective passage orifice (which is normally firmly anchored on the tray) and finally reach a stroke height corresponding to the gas loading (instead of a guide cage, the disk may also possess upwardly movable valve legs anchored to the tray, the upward mobility of which has an upper limit). The gas stream ascending through the passage orifice is deflected at the underside of the raised lid (plate, disk) in a similar manner to that in the bubble cap (in the case of a bubble-cap passage orifice) and exits from the exit region formed under the raised plate (lid, disk) and, as is the case for the bubble-cap tray, enters the liquid backed up on the tray parallel thereto. The plate stroke thus controls the size of the gas exit region and automatically adjusts to the column loading until the upper end of the guide cage limits the maximum possible stroke height. The plates may have spacers directed downward, such that, at low gas loading, the valve closes only to such an extent that the space provided by the spacers still permits vigorous mixing of the horizontal gas outflow with the crossflowing liquid. Spacers also counteract sticking of the valve disk on the tray. Through suitable configuration of the valve elements of a crossflow valve tray, the blowing direction of the valve element can be adjusted, and hence the forced liquid flow on the crossflow valve tray can additionally be influenced (cf., for example, DD 216 633 A1). The principle of crossflow valve trays, and valve trays usable for the purposes of the present document, can be found, for example, in Technische Fortschrittsberichte, volume 61, Grundlagen der Dimensionierung von Kolonnenboden, pages 96 to 138. As well as the above-described moving valves, the person skilled in the art is also aware of fixed valves. These are normally disk-shaped, or trapezoidal, or rectangular units which are punched out of the tray plate and are connected thereto via fixed legs directed upward.

Especially in the case of relatively large diameters of a separating column, on crossflow mass transfer trays, a notable liquid gradient naturally forms proceeding from the at least one feed until attainment of the outlet weir of the at least one outlet (the gradient of the backup height of the liquid feeds the crossflow (to a limited degree)). The result of this is that, in regions with a relatively low liquid height, due to the resulting lower resistances, the ascending vapor (the ascending gas) can pass through the liquid layer more easily in comparative terms. This can ultimately give rise to an inhomogeneous gas loading of the crossflow mass transfer tray (there is preferential flow through the regions with a lower liquid height (a lower flow resistance)), which impairs the separating action thereof. A compensating effect is possible in this respect through the use of, for example, bubble caps of adjustable height (alternatively, the bubble-cap size can also be altered) in the case of crossflow bubble-cap trays, or by use of, for example, plates (lids) with different weight in the case of crossflow valve trays, such that the mass transfer tray produces gas essentially homogeneously over its cross section (where the liquid height on the crossflow mass transfer tray is lower, the height of the bubble cap is, appropriately in application terms, selected at a correspondingly lower level, or the weight of the stroke plate (stroke lid) is selected at a correspondingly higher level; the height of the bubble cap can, for example, also be lowered by controlled shortening of the length of the corresponding chimney, at the end of which the bubble cap is optionally screwed on; alternatively or additionally, for example, the serration/slot structure of the bubble-cap edge can also be varied in order to bring about the desired flow resistance compensation; ideally, the adjustment is made over the crossflow mass transfer tray such that, in operation of the separating column, every bubble cap present on a crossflow bubble-cap tray causes the same flow resistance for the ascending gas). Otherwise, the passages (the passage orifices) of a crossflow mass transfer tray are generally advantageously configured uniformly.

Orifices running (from the top downward) through a crossflow mass transfer tray, the cross-sectional area of which is typically more than 200 times smaller than the overall cross-sectional area of all other orifices of the crossflow mass transfer tray (not including the cross section of the at least one downcomer), do not constitute (separating) passage orifices for the gas ascending through the crossflow mass transfer tray and are therefore not counted as part thereof. For example, such orifices may be tiny emptying holes through which hydraulically sealed crossflow trays can empty when a separating column is shut down. It is also possible for such orifices to serve for screw connection purposes.

Sequences of mass transfer trays having at least one downcomer, in which the at least one feed and the at least one outlet are present, for example, in the same half of the (circular) mass transfer tray, or in which the at least one feed is in the middle of the tray and the at least one outlet is at the edge of the tray, do not constitute a sequence of crossflow mass transfer trays in the sense of the application (of the invention).

The efficacy of crossflow mass transfer trays designed as described is typically less than that of one theoretical plate (one theoretical separation stage). A theoretical plate (or theoretical separation stage) shall be understood in this document quite generally to mean that spatial unit of a separating column which comprises separating internals and is used for a thermal separation process which brings about enrichment of a substance in accordance with the thermodynamic equilibrium. In other words, the term “theoretical plate” is applicable both to separating columns with mass transfer trays and to separating columns with structured packings and/or random packings.

The prior art recommends the use of sequences of at least two identical crossflow mass transfer trays, in separating columns including those comprising separating internals, which are employed for performance of thermal separation processes between at least one gas stream ascending in the separating column and at least one liquid stream descending in the separating column, and wherein at least one of the streams comprises at least one (meth)acrylic monomer. For example, documents DE 19924532 A1, DE 10243625 A1 and WO 2008/090190 A1 recommend the additional use of a sequence of identical hydraulically sealed crossflow mass transfer trays in a separating column for performance of a process for fractional condensation of a product gas mixture comprising acrylic acid from a heterogeneously catalyzed gas phase partial oxidation of C₃ precursors of acrylic acid with molecular oxygen, which comprises, from the bottom upward, at first dual-flow trays and subsequently hydraulically sealed crossflow mass transfer trays.

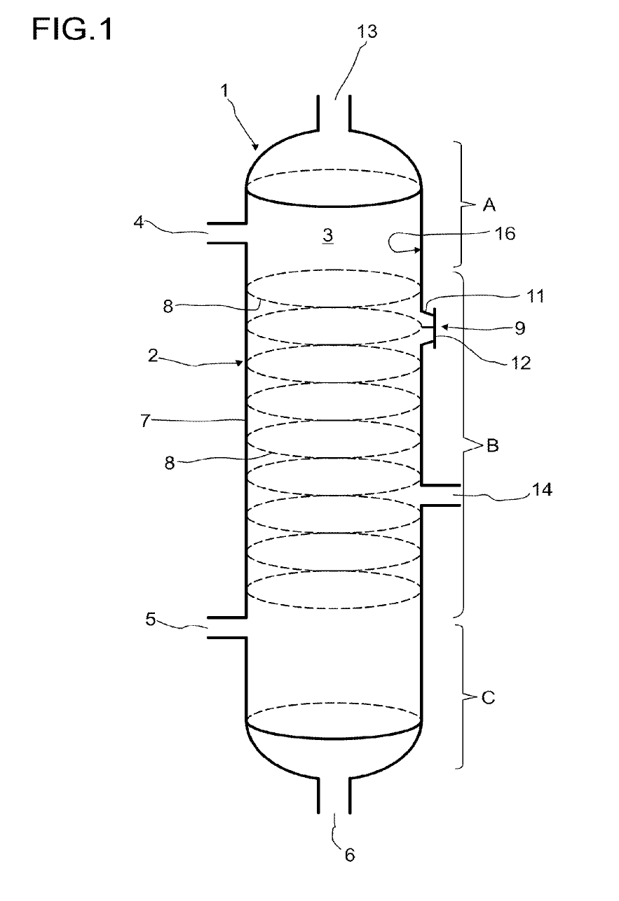

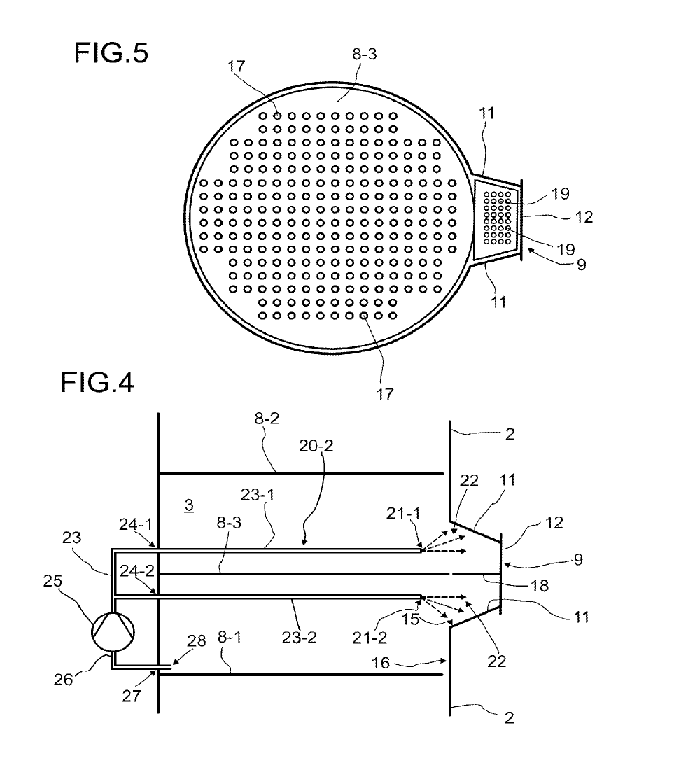

A characteristic feature of the sequences of crossflow mass transfer trays recommended in the prior art is that the lower of two successive crossflow mass transfer trays in the sequence in each case, in the direction of crossflow from the at least one feed thereof to the at least one downcomer thereof, has passage orifices only in the region between the at least one feed and the at least one downcomer (the at least one downflow orifice) (cf., for example, FIGS. 3 and 4 of DE 10243625 A1, FIG. 1 of DD 279822 A1, FIG. 1 of DD 216633 A1, and FIG. 1 from Chemie-Ing.-Techn. Volume 45, 1973/No. 9+10, pages 617 to 620).

The invention relates especially to columns in which the aforementioned trays are used.

A problematic property of (meth)acrylic monomers is the tendency thereof to unwanted polymerization, which cannot completely be suppressed even by the addition of polymerization inhibitors, particularly in the liquid phase.

A disadvantage of known separating columns is that, in the case of continuous performance of the thermal separation process, there is comparatively frequently formation of unwanted polymer over prolonged periods of operation in the mass transfer trays. This is particularly disadvantageous because the operator of the thermal separation process, due to the unwanted polymer formation, has to interrupt the thermal separation process time and again in order to remove the polymer formed. This is because the latter can partly or completely block the passage orifices of the mass transfer tray. Moreover, the free-radical polymerization of (meth)acrylic monomers is normally markedly exothermic, i.e. has high evolution of heat. There is the risk of polymerization proceeding so violently that the separating column comprising the polymerization mixture explodes.

In order to be able to undertake particular inspection operations in the column or in order to clean the column cavity, inspection orifices are typically provided in the column body. Such an inspection orifice is formed, for example, in a stub disposed within the column body. The diameter of the inspection orifice is matched to the intended function of the inspection orifice. Thus, the orifice may be a so-called handhole through which a person can introduce his or her hand, for example together with a cleaning device. In addition, the inspection orifice may be designed as a manhole, where the diameter of the orifice is sufficiently large that, when the column is not in operation, a worker can enter the cavity of the columns, in order to undertake inspection and cleaning operations. Through the inspection orifice, it is also possible, for example, in the course of operation of the column, to remove undesirably formed polymer of acrylic acid.

The trays mounted in the column cavity are typically arranged such that the inspection orifice is between two trays. If the inspection orifice, however, takes the form of a manhole, this has the disadvantage that the distance between the trays becomes undesirably large. If no separating internals are provided in the region of the inspection orifice, unwanted polymer can form in this region.

To solve this problem, WO 2013/139590 A1 proposed mounting separating internals in the manhole region of a condensation column as well and in this way reducing the distance from the transition tray. However, the problem of unwanted polymer forming in the region of the inspection orifice remains.

It is therefore an object of the present invention to provide a column and a thermal separating process of the types specified at the outset, in which polymerization of the material present within the separating column can be prevented or at least reduced.

According to the invention, this object is achieved by a column having the features of claim 1 and a thermal separating process having the features of claim 13. Advantageous configurations and developments are apparent from the dependent claims.

Accordingly, a column has been found for thermal treatment of fluid mixtures, having a cylindrical, vertical column body which forms a column cavity, a plurality of trays mounted in the column cavity and spaced apart from one another, has at least one stub disposed within the column body and extending from the column body, and a closable inspection orifice formed in the stub, it being a characteristic feature of the column that a spray device disposed within the column body can spray liquid at least against the surface of the stub directed into the column cavity, i.e. the inner surface of the stub.

The spatial terms “top”, “bottom”, “horizontal” and “vertical” relate, unless explicitly stated otherwise, to the orientation of the column during operation.

It has been found that unwanted polymer forms, especially in the so-called dead zones in the column. In such dead zones, the residence time of the fluid in the column is particularly long. Such a long residence time promotes polymerization. It has been found that dead zones can form especially in the region of the inspection orifice. According to the invention, this polymer formation can be prevented by spraying the inner surface of the stub with liquid by means of the spray unit during the operation of the column, so as to prevent liquid from dwelling for a longer period at the inner surface of the stub.

According to one design, the spray device has a spray nozzle, an inlet and a spray liquid feed device. The spray liquid feed device is designed to draw spray liquid from the column cavity, to feed the spray liquid withdrawn through the inlet to the spray nozzle and to spray it by means of the spray nozzle at least against the inner surface of the stub. The spray liquid is especially withdrawn above a tray mounted in the column cavity and, more particularly, not from the column bottom. The use of the liquid present in the column cavity as spray liquid results in the advantage that the spray liquid has essentially the same composition as the liquid rinsed away at the inner surface of the stub.

The spray liquid feed device especially has an intake orifice disposed immediately above a tray adjacent to the stub. Preferably, the intake orifice is disposed immediately above the tray disposed directly beneath the stub. In this case, the spray liquid is withdrawn from the column cavity in a region adjacent to the height of the stub, especially directly below the stub. This has the advantage that the spray liquid is withdrawn at the same level in the column, such that the spray liquid and the liquid to be rinsed off at the inner surface of the stub have the same composition. This has a positive effect on the separating effect of a process which is conducted with the column of the invention.

According to a further configuration of the column of the invention, the column body forms a vertical inner surface. In a vertical section of the column, the line of the lower line of intersection of the stub directed into the column cavity, which is part of the surface of the stub, or a tangent to said line of the lower line of intersection of the stub at least in sections forms an angle within a range from 210° to 267° with the vertical inner line of the column body which extends downward from the stub and which is part of the inner surface of the column body. In an advantageous configuration, this angle is within a range from 225° to 267° and preferably within a range from 255° to 267°.

It has been found that dead zones can form especially in the lower portion of the stub. Typically, the lower wall of the stub extends horizontally away from the column body. On this horizontal surface, however, liquid can collect, which dwells for longer in the column. If a polymerizable material is being treated in the column, there is thus unwanted polymer formation on this horizontal surface of the stub of the inspection orifice. According to the invention, this formation of polymer can be prevented by inclining the lower portion of the stub in such a way that liquid which precipitates on the surface of the stub directed into the column cavity runs off back into the column cavity. The angle of inclination should be at least 3′; in this case, the angle of the line of the lower line of intersection of the stub directed into the column cavity with the vertical inner line of the column body which extends downward from the stub is 267°. The inclination is preferably even greater, although excessive inclinations lead to increasing orifice sizes in the column body for the stub. The choice of angle is thus a compromise between a suitable inclination of the lower surface of the stub to the horizontal on the one hand and a suitable diameter of the stub on the other hand.

According to a further configuration of the column of the invention, in the case of a vertical cross section of the column, at least 50% of the line of the lower line of intersection of the stub directed into the column cavity or the tangent to at least 50% of said line of the lower line of intersection of the stub forms an angle within a range from 210° to 267° with the vertical inner line of the column body which extends downward from the stub, preferably an angle within a range from 225° to 267° and more preferably an angle within a range from 255° to 267°. In this case, the inclination, i.e. the angle to the horizontal, of the lower surface of the stub may also be lower in sections, meaning that the aforementioned angle may be greater than the angle specified. Preferably, however, 70%, further preferably 90% and especially 100% of the lower line of intersection of the stub is within the angle range mentioned.

The column stub has an upper half and a lower half. In the column of the invention, especially in the lower half, the surface of the stub directed into the column cavity or the tangent to the surface of the stub forms an angle within a range from 210° to 267° with the vertical inner surface of the column body which extends downward from the stub, preferably an angle within a range from 225° to 267° and more preferably an angle within a range from 255° to 267°. This is because the unwanted polymer forms especially at the surfaces of this lower half of the stub. Said choice of angle relative to the vertical inner surface of the column body prevents liquid from remaining at the surfaces of the lower half of the stub and forming polymer.

Advantageously for application purposes, however, the stub is rotationally symmetric about a horizontal axis. In this case, the entire surface of the stub directed into the column cavity or the tangent to the surface of the stub forms an angle within a range from 210° to 267° with the vertical inner surface of the column body, preferably an angle within a range from 225° to 267° and more preferably an angle within a range from 255° to 267°.

The stub may, for example, be frustoconical. In that case, the surface of the stub directed into the column cavity forms an angle within the aforementioned range with the vertical inner surface of the column body. In the case of a frustoconical stub, liquid precipitating at the surface of the stub can run back into the column cavity particularly efficiently.

According to another configuration of the column of the invention, the stub extends horizontally away from the vertical column body. In this case, polymer formation on the horizontally aligned region of the stub is prevented by means of the spray device.

The stub is especially arranged in vertical direction between two trays mounted in the column cavity. These two trays need not necessarily be adjacent. It is also possible for other trays to be present between these two trays in the region of the stub.

According to a further configuration of the column of the invention, the inspection orifice is a manhole orifice which is formed in the stub and can be closed with a cover. If, in this case, at least one of the trays is mounted in the region of the manhole orifice, it is advantageous for a plate to be disposed in the region of the stub between the one tray and the closed cover. This plate can advantageously prevent ascending gas or descending liquid from flowing through a horizontal orifice in the region of the manhole orifice past the tray mounted in the region of the manhole orifice.

Preferably, the entire cross section of the column body in the region of the stub is essentially filled by the one tray in the region of the manhole orifice and the plate. Only at the joins may orifices remain. If the tray in the region of the manhole orifice is a mass transfer tray having orifices, the plate preferably takes the form of a mass transfer plate. This mass transfer plate especially also has orifices through which gas can ascend and liquid can descend, which results in mass transfer.

The one tray in the region of the manhole orifice and the plate are especially aligned essentially horizontally. The cover may be secured in the stub in a pivotable manner. In addition, the plate may be secured on the cover, such that it is removed when the cover is detached or pivoted away from the stub.

The inspection orifice formed in the stub especially has a circular cross section. However, other round, oval or, less commonly, rectangular cross sections are possible. The clear width of the inspection orifice is within a range from 100 mm to 800 mm. If the inspection orifice takes the form of a manhole orifice, the clear width is especially within a range from 400 mm to 800 mm. Only if the taking of large tools or other large parts through the manhole orifice is envisaged can this orifice be configured to be even larger. If the inspection orifice takes the form of a handhole orifice, the clear width is lower, especially within a range from 100 mm to 300 mm.

The tray which is used in the inventive column is especially a dual-flow tray. In dual-flow trays, there is a particularly high risk of polymerization in the case of use of a fluid mixture comprising (meth)acrylic monomers. By means of the inventive column, in this case, it is possible to reduce the formation of polymer and hence the explosion risk in a particularly effective manner.

However, the trays mounted in the column may also be other trays, as described by way of introduction. Further separating internals may be disposed between the trays. The separating internals improve the mass separation in a column which is used as a separating column.

These further internals may be provided, for example, in the form of packings, especially structured or ordered packings, and/or beds of random packings. Among the random packings, preference is given to those comprising rings, helices, saddles, Raschig, Intos or Pall rings, Berl or Intalox saddles, Top-Pak etc. Structured packings particularly suitable for extraction columns for use in accordance with the invention are, for example, structured packings from Julius Montz GmbH in D-40705 Hilden, for example the Montz-Pak B1-350 structured packing. Preference is given to using perforated structured packings made from stainless steel sheets. Packed columns having ordered packings are known per se to those skilled in the art and are described, for example, in Chem.-Ing. Tech. 58 (1986) no. 1, pages 19-31 and in the Technische Rundschau Sulzer 2/1979, pages 49 ff. from Gebrüder Sulzer Aktiengesellschaft in Winterthur, Switzerland.

The inventive column can especially be used as a separating column. The separating column has a sequence of trays. The clear distance between two immediately successive trays within the inventive column is especially not more than 700 mm, preferably not more than 600 mm or not more than 500 mm. Appropriately in application terms, the clear distance within the tray sequence is 300 to 500 nm. In general, the tray separation should not be less than 250 mm.

The height of the column body is, for example, greater than 5 m, especially greater than 10 m. However, it is also possible for the height of the column body to exceed 30 m or 40 m.

The invention further relates to a thermal separating process between at least one gas ascending within a column, as described above, and at least one liquid descending within the column. In this case, the ascending gas and/or the descending liquid especially comprises (meth)acrylic monomers.

The thermal separating process according to the invention may, for example, be a process for fractional condensation for separation of acrylic acid from a product gas mixture comprising acrylic acid from a heterogeneously catalyzed gas phase oxidation of a C₃ precursor compound (especially propene and/or propane) of the acrylic acid with molecular oxygen to give acrylic acid.

The separating column (condensation column) may be configured as described in documents DE 10243625 A1 and WO 2008/090190 A1.

There follows an elucidation of working examples of the inventive column and working examples of the process according to the invention with reference to the drawings.

FIG. 1 shows a schematic view of a column in a working example of the invention,

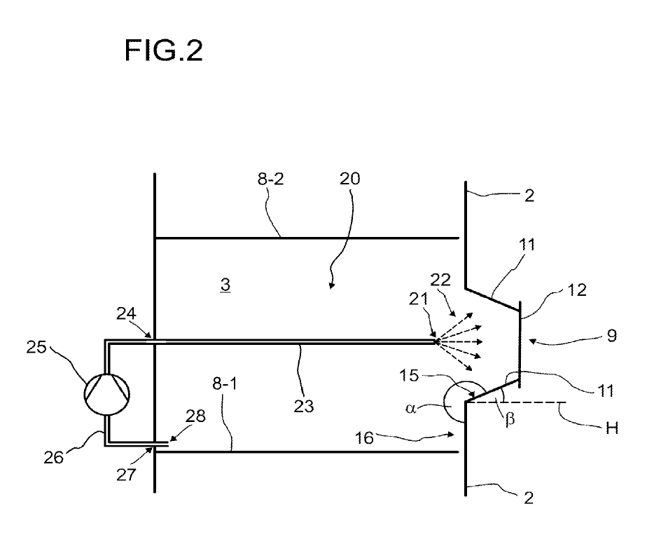

FIG. 2 shows a detail of a vertical cross section of the column shown in FIG. 1 in the region of an inspection orifice,

FIG. 3 shows a detail of a vertical cross section of a further working example of the column of the invention,

FIG. 4 shows a detail of a vertical cross section of yet a further working example of the column of the invention and

FIG. 5 shows a horizontal cross section of the column shown in FIG. 4 in the region of the inspection orifice.

The working example described hereinafter relates to a separating column 1 as used, for example, in a process for fractional condensation for separation of acrylic acid from a product gas mixture comprising acrylic acid from a heterogeneously catalyzed gas phase partial oxidation of a C₃ precursor compound (especially propene and/or propane) of the acrylic acid with molecular oxygen to give acrylic acid.

FIG. 1 shows the separating column 1 known per se in schematic form. It comprises a cylindrical column body 2, the axis of which is aligned vertically. The column body 2 is essentially a hollow cylinder. This means that the shell 7 of the column body 2 forms a column cavity 3. The column body 2 is manufactured from stainless steel. On the outside, the separating column 1 is normally thermally insulated in a conventional manner. The height of the separating column 1 is 40 m. The internal diameter of the shell 7 of the column body 2 is 7.4 m throughout.

In the vertical direction, the separating column 1 is divided into three regions: the upper region A is referred to as the column head. At the column head is provided a feed 4 through which a liquid can be introduced into the column cavity 3. In addition, an offgas line 13 for withdrawal of the gaseous mixture is formed at the top.

Beneath the column head, a region B is formed. In this region, the fractional condensation is conducted. A withdrawal line 14 is disposed within this region, through which crude acrylic acid is withdrawn.

Beneath region B, the column bottom is formed in region C. In the column bottom, there is an inlet 5 for introduction of the product gas mixture into the column cavity 3. In addition, there is an outlet 6 for the bottoms liquid in the column bottom.

In region B, several trays 8 are secured in the column cavity 3. The trays 8 of the column 1 are horizontal and are mounted with vertical spacing in the column cavity 3. This forms horizontal surfaces facing downward in the trays 8. The trays 8 serve as separating internals which improve separation in the separating column 1. The trays 8 are dual-flow trays. It is also possible to use other trays among those mentioned by way of introduction.

In order to be able to undertake inspection and cleaning operations when the column 1 is not in operation, at least one inspection orifice 9 is formed in the column body 2. For this purpose, the shell 7 or the column body 2 has an orifice. The cross section of the orifice is circular. If required, however, other cross-sectional shapes may also be used. At the edge of this orifice is secured a frustoconical stub 11 in a liquid- and gas-tight manner. The axis of symmetry of the stub 11 is aligned horizontally, such that the stub 11 extends away from the column body 2. The end of the stub 11 pointing away from the column body 2 forms the inspection orifice 9. At this end, a cover 12 is also provided. The cover 12 is secured in the stub 11 so as to be pivotable. In the closed state, the cover 12 closes the inspection orifice 9 in a liquid- and gas-tight manner. In the pivoted-open state of the cover 12, the column cavity 3 is accessible from outside via the inspection orifice 9.

FIG. 1 shows only one stub 11. Typically, the common body 2 comprises several stubs 11 spaced apart in vertical direction with the corresponding inspection orifices 9.

The diameter of the inspection orifice 9 is guided by the purpose of the inspection orifice 9. In the working example described here, the inspection orifice 9 takes the form of a manhole orifice. The diameter of this manhole orifice is within a range from 400 mm to 800 mm.

FIG. 2 shows the configuration of the inspection orifice 9 in detail. The column body 2 has a vertical inner surface 16 aligned into the column cavity 3. In addition, the stub 11 also has a surface 15 directed into the column cavity 3. This is the inner surface of the stub 11.

Below and above the inspection orifice 9 is disposed a mass transfer tray 8-1 and 8-2. Since the inspection orifice 9 is a manhole orifice, the distance between these two mass transfer trays 8-1 and 8-2 is relatively large, for example 1000 mm. This relatively large distance between the two mass transfer trays 8-1 and 8-2 can lead to unwanted polymer formation. In order to prevent polymerization in the region between the mass transfer trays 8-1 and 8-2, especially in the stub 11, a spray device 20 is disposed in the column body 2. By means of the spray device 20, it is possible to spray a liquid 22 at least against the surface 15 of the stub 11 directed into the column cavity 3. For this purpose, the spray device 20 has a spray nozzle 21 which is fed with liquid via an inlet 23. The inlet 23 passes through the column body 2 through a gas- and liquid-tight leadthrough 24. Outside the column body 2 is disposed a pump 25 connected to the inlet 23. On the other side, the pump 25 is connected to a line 26 which enters the column cavity again through a further gas- and liquid-tight leadthrough 27. The line 26 has an intake orifice 28 is disposed immediately above the mass transfer tray 8-1. In this case, the mass transfer tray 8-1 is adjacent to the inspection orifice 9 and the stub 11.

In the working example illustrated here, this mass transfer tray 8-1 is immediately below the inspection orifice 9. By means of the spray device 20, liquid which has collected on the mass transfer tray 8-1 is withdrawn and sprayed by the spray nozzle 21 against the surface 15 of the stub 11 and the inner surface of the cover 12. This prevents liquid from collecting and polymerizing in this region.

In the vertical cross section of the column 1 shown in FIG. 2, the surface 15 of the lower line of intersection of the stub 11, which is shown as a line in FIG. 2 because of the sectional representation, forms the angle a with the vertical inner surface 16 of the column body 2 which extends downward from the stub 11 and which is also shown as a line in FIG. 2 because of the sectional representation. At the vertex of the angle, the vertical inner surface 16 of the column body 2 and the lower line of intersection of the stub 11 are thus connected. Correspondingly, this surface 15 of the stub 11 forms the angle 13 with the horizontal H, the sum of the angles a and 13 being 270°.

In the configuration shown in FIG. 2, the angle 13 is greater than 0, meaning that the surface 15 in the case of the lower line of intersection of the vertical cross section of the column 1 is not aligned horizontally but inclined. The angle of inclination in the present working example is 3°, although the drawings are not a true reproduction of the angles for better illustration. The angle a in this case is thus 267°.

It is pointed out that the angle a may also be smaller, resulting in a more significant inclination of the surface 15. The angle a is, for example, within a range from 210° to 267°, especially within a range from 225° to 267° and preferably within a range from 255° to 267°.

The inclination of the surface 15 of the lower line of intersection of the stub 11 in the case of a vertical cross section of the column 1 has the effect that liquid on the surface 15 runs off downward and especially does not remain on this surface 15. In this way, it is possible to prevent the polymerization of liquid comprising (meth)acrylic monomers.

The inclination of at least 3° is advantageous especially in the lower region of the stub 11, in order that liquid can run off. More particularly, the lower half of the stub 11 is at this angle to the inner surface 16 of the column body 2. For manufacturing reasons, however, the stub 11 is preferably rotationally symmetric, such that the angle between the surface of the stub 11 directed into the column cavity 3 and the inner surface 16 of the column body 2 is the same over the entire circumference of the stub 11. In terms of cross section, the inner line which is part of the inner surface 15 of the stub 11 is a straight line. In other working examples, however, this line may also be curved. In this case, for the angle α or the angle β, the tangent to the surface 15 of the lower line of intersection of the stub 11 with the vertical inner surface 16 of the column body 2 is considered. In the case of a curved line, the alignment of these tangents changes. The above-specified angle α in this case is within the angle range specified at least in 50% and preferably over a greater region, for example 70% or 90%. The angle α is especially not 270° or greater in any region.

In the working example of the column 1 of the invention shown in FIG. 3, the angle a is 270°, meaning that the surface 15 in this case is aligned horizontally at the lower line of intersection of the vertical cross section of the column 1 and not inclined as in the working example shown in FIG. 2. The stub 11 is cylindrical. However, polymer formation is prevented in this working example as well by the spray device 20.

With reference to FIGS. 4 and 5, a further working example of the column 1 of the invention is described:

As in the working example shown in FIGS. 1 to 3, the inspection orifice 9 is a manhole orifice. The distance between the mass transfer trays 8-1 and 8-2 is relatively large in this case, for example 1000 mm. This relatively large distance between the two mass transfer trays 8-1 and 8-2 can lead to unwanted polymer formation. For this reason, in the working example of FIGS. 4 and 5, a mass transfer tray 8-3 is also disposed in the region of the inspection orifice 9. The distance between the two mass transfer trays 8-1 and 8-3 and between the two mass transfer trays 8-3 and 8-2 in that case is 500 mm. The mass transfer tray 8-3 in the working example described is a dual-flow tray having orifices 17, as shown in FIG. 5.

Additionally disposed in the region of the inspection orifice 9 is a plate 18 which prevents ascending gas in particular, but also descending liquid, from flowing upward or downward past the mass transfer tray 8-3 through the horizontal orifice formed by the stub 11. The plate 18 has orifices 19, such that it acts as a mass transfer plate. The plate 18 is aligned horizontally, flush with the mass transfer tray 8-3. The plate 18 is thus disposed horizontally at the same level as the mass transfer tray 8-3. The shape of the plate 18, as shown in FIG. 3, is matched to the horizontal cross-sectional shape of the stub. Since the stub in the present working example is frustoconical, the plate 18 is trapezoidal. In order to keep the gap or join between the plate 18 and the mass transfer tray 8-3 as narrow as possible, the long edge of the trapezium of the plate 18 could also be matched to the rounding of the mass transfer tray 8-3 in this region or, conversely, the rounding of the mass transfer tray 8-3 in this region could be truncated to match the long edge of the trapezoidal plate 18.

According to the size of the inspection orifice 9 and the desired distance between the mass transfer trays 8, it is also possible for several plates 8 to be present in the region of the inspection orifice 9. In that case, one plate 18 is assigned to each of these mass transfer trays 8.

The plate 18 is secured on the pivotable cover 12, such that it is pivoted away with the cover 12 when the inspection orifice 9 is opened. This has the advantage that the plate 18 need not be detached when inspection or cleaning operations have to be conducted in the column 1. Equally, the mass transfer tray 8-3 is also detachable, such that a person can get through the inspection orifice 9 in the form of a manhole into the column cavity 3.

As shown in FIGS. 4 and 5, the stub 11 is frustoconical as in the previous working examples. Alternatively, however, it could also be cylindrical as shown in FIG. 3.

The spray device 20-2 of the working examples of FIGS. 4 and 5 differs from the spray unit 20 of the above-described working examples in that the inlet 23 branches into an upper inlet 23-1 and a lower inlet 23-2 which then enter the column cavity 3 through the leadthroughs 24-1 and 24-2. The upper inlet 23-1 is disposed above the tray 8-3, and the lower inlet 23-2 below the tray 8-3. The upper inlet 23-1 opens into a spray nozzle 21-1, by means of which liquid 22 is sprayed against the upper surface 15 of the stub 11 and the inner surface of the cover 12. The lower inlet 23-2 opens into a spray nozzle 21-2, by means of which liquid 22 is sprayed against the lower surface 15 of the stub 11 and the inner surface of the cover 12.

In this working example too, a pump 25 connected to the inlet 23 is disposed outside the column body 2. On the other side, the pump 25 is connected to the line 26 which enters the column cavity again via a further gas- and liquid-tight leadthrough 27. The intake orifice 28 of the line 26 in this case is disposed directly above the mass transfer tray 8-1. The mass transfer tray 8-1 here is the closest mass transfer tray beneath the stub 11.

In further working examples, the spray device 20, proceeding from the inlet 23, may also comprise a line system which sprays the inner surfaces of further inspection orifices with liquid. In this case, the composition of the liquid withdrawn via the intake orifice 28, however, is not always essentially the same as the composition of the liquid in the region of the respective inspection orifice 9 in the operation of a separation process where, more particularly, gas ascends and a liquid descends.

There follows a description of a working example of the process according to the invention which is executed with the above-described separating column 1.

The process is a thermal separating process between at least one gas ascending in the separating column 1 and at least one liquid descending in the separating column 1. The ascending gas and/or the descending liquid especially comprises (meth)acrylic monomers.

In the separation process, a fractional condensation for separation of acrylic acid from a product gas mixture comprising acrylic acid from a heterogeneously catalyzed gas phase partial oxidation of a 0₃ precursor compound (especially propene and/or propene) of the acrylic acid with molecular oxygen to give acrylic acid is conducted in a separating column 1 comprising separating internals. The separating column comprises, from the bottom upward, first dual-flow trays and then crossflow capped trays, which are supported from beneath as described above. Otherwise, the process is conducted as described in documents DE 19924532 A1, DE 10243625 A1 and WO 2008/090190 A1.

The term “C₃ precursor” of acrylic acid encompasses those chemical compounds which are obtainable in a formal sense by reduction of acrylic acid. Known C₃ precursors of acrylic acid are, for example, propane, propene and acrolein. However, compounds such as glycerol, propionaldehyde, propionic acid or 3-hydroxypropionic acid should also be counted among these C₃ precursors. Proceeding from these, the heterogeneously catalyzed gas phase partial oxidation with molecular oxygen is at least partly an oxidative dehydrogenation. In the relevant heterogeneously catalyzed gas phase partial oxidations, the C₃ precursors of acrylic acid mentioned, generally diluted with inert gases, for example molecular nitrogen, CO, CO₂, inert hydrocarbons and/or water vapor, are passed in a mixture with molecular oxygen at elevated temperatures and optionally elevated pressure over transition metal mixed oxide catalysts, and converted oxidatively to a product gas mixture comprising acrylic acid.

Typically, the product gas mixture comprising acrylic acid from a heterogeneously catalyzed gas phase partial oxidation of C₃ precursors (e.g. propene) of acrylic acid with molecular oxygen over catalysts in the solid state, based on the total amount of the specified constituents present (therein), has the following contents:

1% to 30% by weight of acrylic acid,

0.05% to 10% by weight of molecular oxygen,

1% to 30% by weight of water,

0% to 5% by weight of acetic acid,

0% to 3% by weight of propionic acid,

0% to 1% by weight of maleic acid and/or maleic anhydride,

0% to 2% by weight of acrolein,

0% to 1% by weight of formaldehyde,

0% to 1% by weight of furfural,

0% to 0.5% by weight of benzaldehyde,

0% to 1% by weight of propene, and

as the remainder, inert gases, for example nitrogen, carbon monoxide, carbon dioxide, methane and/or propane.

The partial gas phase oxidation itself can be performed as described in the prior art. Proceeding from propene, the partial gas phase oxidation can be performed, for example, in two successive oxidation stages, as described, for example, in EP 700 714 A1 and in EP 700 893 A1. It will be appreciated, however, that it is also possible to employ the gas phase partial oxidations cited in DE 19740253 A1 and in DE 19740252 A1.

In general, the temperature of the product gas mixture leaving the partial gas phase oxidation is 150 to 350° C., frequently 200 to 300° C.

Direct cooling and/or indirect cooling cools the hot product gas mixture appropriately at first to a temperature of 100 to 180° C., before it is conducted, for the purpose of fractional condensation, into region C (the bottom) of separating column 1. The operating pressure which exists in the separation column 1 is generally 0.5 to 5 bar, frequently 0.5 to 3 bar and in many cases 1 to 2 bar.

### LIST OF REFERENCE NUMERALS

1 column, separating column

2 column body

3 column cavity

4 feed

5 inlet

6 outlet

7 shell

8 trays

8-1, 8-2, 8-3 trays

9 inspection orifice

11 stub

12 cover

13 draw point

14 withdrawal line

15 surface

16 inner surface

17 orifice

18 plate

19 orifice

20 spray device

21 spray nozzle

22 liquid

23 inlet

24 leadthrough

25 pump

26 line

27 leadthrough

28 withdrawal orifice

1. A column for thermal treatment of fluid mixtures, the column comprising:
a cylindrical, vertical column body which forms a column cavity,
a plurality of trays mounted in the column cavity and spaced apart vertically from one another,
at least one stub disposed within the column body and extending away from the column body, and
a closable inspection orifice formed in the stub,
wherein
a spray device disposed in the column body can spray liquid at least against the surface of the stub directed into the column cavity.

2. The column according to claim 1, wherein the spray device has a spray nozzle, an inlet a spray liquid feed device, the spray liquid feed device being designed to draw spray liquid from the column cavity, to feed the spray liquid withdrawn through the inlet to the spray nozzle and to spray it by means of the spray nozzle at least against the surface of the stub directed into the column cavity.

3. The column according to claim 1, wherein the spray liquid feed device has an intake orifice disposed immediately above a tray adjacent to the stub.

4. The column according to claim 1, wherein:
the column body forms a vertical inner surface:, and
in the case of a vertical cross section of the column the line of the lower line of intersection of the stub directed into the column cavity or a tangent to the line of the lower line of intersection of the stub directed into the column cavity at least in sections forms an angle within a range from 210° to 267° with the vertical inner line of the column body.

5. The column according to claim 4, wherein in the case of a vertical cross section of the column at least 50% of the line of the lower line of intersection of the stub directed into the column cavity or the tangent to 50% of the line of the lower line of intersection of the stub directed into the column cavity forms an angle within a range from 225° to 267° with the vertical inner line of the column body.

6. The column according to claim 4, wherein the stub has an upper half and a lower half and in the lower half the surface of the stub directed into the column cavity or the tangent to the surface of the lower half of the stub forms an angle within a range from 210° to 267° with the vertical inner surface of the column body.

7. The column according to claim 4, wherein the stub is rotationally symmetric about a horizontal axis and the surface of the stub directed into the column cavity or the tangent to the surface of the stub forms an angle within a range from 210° to 267° with the vertical inner surface of the column body.

8. The column according to claim 4, wherein the stub is frustoconical and the surface of the stub directed into the column cavity forms an angle within a range from 210° to 267° with the vertical inner surface of the column body.

9. The column according to claim 1, wherein:
the inspection orifice is a manhole orifice which is formed in the stub and can be closed with a cover,
at least one of the trays is mounted in the region of the manhole orifice; and
a plate is disposed in the region of the stub between the one tray and the closed cover.

10. The column according to claim 9, wherein the entire horizontal cross section of the column at the height of the one tray in the region of the manhole orifice is essentially filled by the one tray and the plate.

11. The column according to claim 9, wherein the one tray in the region of the manhole orifice is a mass transfer tray having orifices and the plate is a mass transfer plate having orifices.

12. The column according to claim 9, wherein the one tray in the region of the manhole orifice and the plate are aligned essentially horizontally.

13. A thermal separation process between at least one gas ascending within the column according to claim 1 and at least one liquid descending within the column.

14. The process according to claim 13, wherein the ascending gas, the descending liquid, or both, comprises (meth)acrylic monomers.
